(19) United States
(12) Patent Application Publication
(10) Pub. No.: US 2006/0155194 A1
(43) Pub. Date: Jul. 13, 2006

(54) Method for detecting occlusions and leakages in subcutaneous blood vessels

(76) Inventors: Ronald Marcotte, New Gloucester, ME (US); Milton Waner, New York, NY (US); Walter Hebold, Raymond, ME (US)

Correspondence Address: Lawson & Persson, P.C., Suite 103, 67 Water Street, Laconia, NH, 03246, US

(21) Appl. No.: 11/035,397

(22) Filed: Jan. 13, 2005

(51) Int. Cl. A61B 6/00

(52) U.S. Cl. 600/473

(57) ABSTRACT

Methods for detecting occlusions and leakages in subcutaneous blood vessels. The method is performed using an infrared imaging system having at least one infrared emitter, an infrared detector, a computing unit, a display, and a power source. The method includes preparing a body target area, supplying power from to the system components, accessing a target blood vessel, introducing a substance into the vessel, locating the vessel such that images of the vessel are captured by the infrared detector and displayed on the display; and examining flow patterns of the substance through the vessel by viewing the images on the display.

### FIELD OF THE INVENTION

The present invention relates to the detection of the presence of occlusions and leakages in subcutaneous blood vessels. In particular, the present invention relates to improved methods for detecting the presence of occlusions and leakages in subcutaneous blood vessels through the aid of dyes or other substances injected into such blood vessels.

### BACKGROUND OF THE INVENTION

Blood vessel occlusions and leakages are responsible for many serious, and often life threatening, pathologies.

Occlusions, which commonly take the form of atherosclerotic vessel disease caused primarily by plaque build-ups on blood vessel walls or foreign obstructions loose in the blood stream, can reduce or eliminate the flow of blood to critical organs within a body, thereby causing illness, disability and death. For example, arteries supplying blood to the brain may become blocked and cause what is commonly referred to as a stroke, while blockages of the arteries supplying blood to the heart may result in heart attack. It is noted that heart attacks and strokes are only two of the major disorders associated with blocked or narrowed blood vessels. These disorders are not easily identified and often occur without noticeable symptoms; requiring regular medical check-ups be performed in order to detect their presence.

Leakage from blood vessels, commonly referred to as “internal bleeding” is also a serious condition and, if left undetected, can result in severe blood loss and the onset of shock. The precise location and extent of leakage from a blood vessel is also difficult to detect, and invasive procedures for proper assessment and repair are typically required.

Traditional diagnostic procedures for blood vessel occlusions or leakages rely on the injection of radio-opaque dyes into the target blood vessel(s) and the use of non-invasive-x-ray or magnetic resonance imaging, or invasive imaging using endoscopes or arthroscopic imaging, to create images of the dye's flow pattern. In the case of MRI diagnostics, this procedure requires that the patient undergo lengthy and uncomfortable examinations inside a large MRI apparatus. In the case of x-ray fluoroscopy diagnostics, patients are subjected to dozens, if not hundreds, of x-ray exposures over long periods, which thereby elevates their radiation exposure levels in a slow and expensive procedure. In the case of endoscopic or arthroscopic imaging, such as those described in connection with U.S. Pat. No. 6,351,663, the patient may experience extreme discomfort, requiring sedation. Further, traditional endoscopes and arthroscopes are only effective at viewing blood vessels on the surfaces of internal tissues and cannot penetrate those tissues to view blood vessels located within said tissues.

Another drawback of traditional dye based diagnostic systems is the difficulty in quickly and accurately identify the target blood vessel(s) and gain IV access with a minimum of physical and emotional trauma to the patient. This difficulty is exacerbated in cases in which dyes must be introduced into less prominent blood vessels as these less prominent blood vessels cannot be found easily by visual and tactile clues, and accessing them may require multiple sticks to the patient, which thereby causes the patient physical and emotional pain and trauma. Inhibited IV access and diagnostic procedures can also subject medical practitioners to legal liability risk, by contributing to the complications associated with improper, ineffective, or delayed IV access and diagnosis. Accordingly, it is clear that current dye based systems for diagnosing blood vessel occlusions and leakages have significant drawbacks.

Recently, a number of other patents have issued that address the diagnosis of occlusions. One such patent is U.S. Pat. No. 6,735,462, which purports to disclose an apparatus for infrared imaging in small passageways. The apparatus includes a catheter that includes an integral infrared imaging system. In use, the catheter is inserted within a blood vessel and takes thermal images of the inside of the vessel using far infrared light in order to identify thermal abnormalities, such as is present in when inflamed lesions are present in these vessels. The apparatus described in this patent does not require the use of X-ray or MRI imaging, and allows blood vessels located within tissues to be observed. However, this device also has significant drawbacks. First, the device is completely ineffective at detecting leakages from blood vessels, as such leakages do not produce any thermal signature. Second, the device is ineffective at detecting partial occlusions caused by plaque or other substances that do not produce a distinct thermal signature. Finally, the device suffers from the same difficulty in quickly and accurately identify the target blood vessel(s) as described above in connection with traditional dye based diagnostic systems.

Another recently issued patent is U.S. Pat. No. 6,780,159, which purports to disclose a system and method of detecting a vascular condition within a body by analyzing vibrations emitted in response to the velocity of blood flowing through a vascular structure. The system disclosed in this patent is substantially non invasive and, therefore, does not require the accurate and rapid penetration of the surface of the skin with an hypodermic needle or catheter that is required in the other patents describe herein. It also does not require the use of X-ray or MRI imaging, or the use of invasive endoscopes or arthroscopes to perform its diagnostic function. Finally, it appears to be usable to detect occlusions in blood vessels located within surrounding tissue. Accordingly, it appears to have a number of advantages. However, it also has a number of disadvantages that make it unsuitable at solving the problems solved by the system and method of the present invention.

First, it relies upon vibrations relating to differences in blood velocity in areas of blood vessels that are not occluded and those that are occluded. Therefore, it is ineffective at diagnosing leakages from blood vessels, as such leakages are unlikely to create any noticeable difference in the velocity of the blood through the vessel. Second, although it may be used to track a narrowing of vessels over time, it is ineffective at first test diagnosis of a general narrowing of blood vessels, as the device requires a comparison of velocities to determine differences. Finally, it is ineffective at diagnosing blockages in less prominent blood vessels, or those further below the surface of the skin, as the vibrations emitted from these less prominent blood vessels are relatively low and are hidden by those emitted form larger, more prominent, vessels.

Therefore, there is a need for an improved system and method that are capable of detecting and diagnosing both blood vessel occlusions and leakages, that allows blocked or leaking blood vessels to be accurately and rapidly located, that allows blocked or leaking blood vessels to be easily located in difficult conditions and body types (e.g., obese patients, dark pigmentation skin, neonates, collapsed veins, low lighting), that reduces patient pain and trauma, both emotionally and physically, that does not require the use of expensive and potentially hazardous x-ray or magnetic resonance imaging devices to analyze flow patterns through the vessels, that does not require the or invasive imaging equipment, such as endoscopes or arthroscopic imaging, that is capable of viewing dye patterns within vessels that are surrounded by tissue, that is effective at detecting partial occlusions caused by plaque or other substances that do not produce a distinct thermal signature, that it is effective at diagnosing blockages in less prominent blood vessels, and that allows minimally trained medical staff to identify and diagnose blood vessel blockages and leakages.

### SUMMARY OF THE INVENTION

The present invention is a method for detecting occlusions and leakages in subcutaneous blood vessels that overcomes the drawbacks inherent in prior art methods. The method is performed with the aid of an infrared imaging system that includes at least one infrared emitter, an infrared detector, a computing unit in communication with the infrared detector, a display in communication with the computing unit, and a power source. In its most basic form, the method includes the steps of preparing a body target area, and supplying power from the power source to the infrared emitter, infrared detector, computing unit, and display of the imaging system such that infrared light is emitted by the infrared emitter, reflected infrared light is received by the infrared detector and converted into signals sent to the computing unit, the computing unit accepts the signals and outputs image data to the display, and the display displays the images. The basic method also includes the steps of accessing a target blood vessel, introducing an IR-opaque substance into the target blood vessel, locating the target blood vessel such that images of the target blood vessel are captured by the infrared detector and displayed on the display; and examining flow patterns of the IR-visible substance through the target blood vessel by viewing the images of the target blood vessel on the display of the imaging system.

In embodiments in which the system is only used in connection with the locating and examining steps, the accessing and introducing steps may be performed before or after the step of supplying power to the components of the system. However, in embodiments in which the system is also used in connection with the accessing step, the step of supplying power to the system is performed prior to the accessing step.

In the preferred method, the step of introducing an IR-visible substance into the target blood vessel includes introducing an IR-opaque substance into the target blood vessel. This introduction may be performed via injection through a hypodermic needle, an intravenous drip administered through a cannula or other art recognized device for administering fluids intravenously, or using other art recognized methods for introducing substances into blood vessels. The preferred IR-visible substance is an IR-opaque substance. The preferred IR-opaque substance is indocyanine green due to its broad acceptance for use in a wide range of human medical procedures. However, other art recognized substances that enhance visibility under infrared light and are accepted for use in human medical treatments may be substituted to achieve similar results

In the preferred method for determining the presence of an occlusion of the blood vessel, the step of examining flow patterns involves examining images displayed on the display to determine the presence of an occlusion by observing an absence of IR-opaque substance through the target blood vessel.

In the preferred method for determining the presence of leakage from the blood vessel, the step of examining flow patterns involves examining images displayed on the display to determine the presence of an leakage by observing the IR-opaque substance flowing outside of the target blood vessel.

In the preferred method for determining the presence of partial occlusion, or narrowing, of the blood vessel, the step of examining flow patterns involves examining images displayed on the display to determine the presence of an narrowing by observing a restricted flow of the IR-opaque substance through a particular area of the target blood vessel.

The preferred method utilizes an imaging system in which the computing unit enhances images of the target blood vessel before outputting the images to the display. In such a method, the locating step is performed before the accessing step and the accessing step includes the step of viewing an enhanced image of the target blood vessel on the display of the imaging system and piercing the target blood vessel with the aid of the enhanced image. In such methods it is also preferred that the locating step include the steps of directing incident light from the infrared emitters on a target area of a surface of a skin and viewing the enhanced image of blood vessels located beneath the target area on the display.

In some variations of the method, the display of the imaging system includes an optical lens disposed between the display and an eye of a user. When such an imaging system is utilized, the locating step may include the steps of viewing the unenhanced image on the target area of the skin, and adjusting the optical lens to correct the enhanced image displayed on the display for depth perception differences between the enhanced image and the unenhanced image. In other variations of the method, the step of locating a target blood vessel includes the steps of viewing the unenhanced image on the target area of the skin, adjusting the display to correct the enhanced image displayed on display for depth perception differences between the enhanced image and the unenhanced image.

The imaging system utilized in the performance of the preferred method includes a data input and a computing unit having a digital signal processor and a memory. When performed with such an imaging system, the step of optimizing the system preferably includes the step of using the data input to specify an enhancement algorithm stored in memory to be used by the digital signal processor to generate the enhanced image. The enhancement algorithm may be selected based upon a body type, pigmentation, and/or age of the patient, or characteristics of the IR-visible substance introduced into the target blood vessel. When selected based upon the characteristics of the substance, the algorithm may enhance the image by targeting wavelengths of light specific to the substance. In some embodiments, the optimizing step includes the step of using the data input to adjust an intensity level of, and/or wavelength of light produced by, the infrared emitter or emitters.

The imaging system used in the preferred method also includes a headset to which the infrared emitter, infrared detector, computing unit, and display are attached. When such embodiments of the system are used, the method also includes the step of disposing the headset on a head of a user and removing the headset once the procedure is completed.

Some embodiments of the method also include the step of adjusting the system after the introducing step has been performed. When the preferred imaging system is used, the step of adjusting the system includes the step of using the data input to specify an enhancement algorithm stored in memory to be used by the digital signal processor to generate the enhanced image and or using the data input to alter the mode operation of the infrared emitters to, for example, vary the intensity or wavelength of light produced thereby. In still other embodiments, the adjusting step includes using the data input to alter parameters of the display.

In embodiments utilizing an imaging system having data storage means for storing multiple images, the method preferably includes the step of recording a sequence of images showing a flow pattern on the data storage means. In some such embodiments, the imaging system includes a digital signal processor programmed with an algorithm to adjust the playback of the sequence of enhanced images stored in the data storage means, which may indicate flow patterns, and the method includes the steps of adjusting the playback of the sequence of enhanced images.

It is noted that the method is not limited to diagnosis and monitoring of occlusions using the preferred system, but rather may be performed using any IR imaging system that includes at least one infrared emitter an infrared detector, a computing unit, a display device, and a power source. Due to the injection of a highly visible substance within the blood vessel, and the fact that the step of examining flow patterns does not require that real time images be provided to the display, the imaging system used to perform the method may not enhance images, or provide images to the display in substantially real time. Therefore, in these embodiments, the steps relating to the use of the system prior to the introduction of the IR-visible substance may be omitted, and the locating may be performed after the blood vessel has been accessed and the IR-visible substance has been injected.

Therefore, it is an aspect of the invention to provide an improved system and method for locating both blood vessel occlusions and leakages.

It is a further aspect of the invention to provide an improved system and method for locating blood vessel occlusions and leakages that increase the speed of vascular disorder diagnosis.

It is a further aspect of the invention to provide an improved system and method for locating blood vessel occlusions and leakages that increases the accuracy of vascular disorder diagnosis.

It is a further aspect of the invention to provide an improved system and method for locating blood vessel occlusions and leakages that reduces patients' physical and emotional pain and trauma associated with IV access and vascular disorder diagnosis.

It is a further aspect of the invention to provide an improved system and method for locating blood vessel occlusions and leakages the does not require the use of expensive and potentially hazardous x-ray or magnetic resonance imaging devices to analyze flow patterns through the vessels.

It is a further aspect of the invention to provide an improved system and method for locating blood vessel occlusions and leakages that does not require the or invasive imaging equipment, such as endoscopes or arthroscopic imaging.

It is a further aspect of the invention to provide an improved system and method for locating blood vessel occlusions and leakages that is capable of viewing dye patterns within vessels that are surrounded by tissue.

It is a further aspect of the invention to provide an improved system and method for locating blood vessel occlusions and leakages that is effective at detecting partial occlusions caused by plaque or other substances that do not produce a distinct thermal signature.

It is a further aspect of the invention to provide an improved system and method for locating blood vessel occlusions and leakages that it is effective at diagnosing blockages in less prominent blood vessels.

It is a further aspect of the invention to provide an improved system and method for locating blood vessel blockages and leakages that allows a minimally trained medical practitioner to locate and monitor vascular disorders, such as obstructions, occlusions, and leakages.

It is a still further aspect of the invention to provide an improved system and method for locating blood vessel blockages and leakages that allows blockages and leakages to be more easily located in difficult conditions and body types (e.g., obese patients, dark pigmentation skin, neonates, collapsed veins, low lighting).

These aspects of the invention are not meant to be exclusive and other features, aspects, and advantages of the present invention will be readily apparent to those of ordinary skill in the art when read in conjunction with the following description, appended claims and accompanying drawings.

### BRIEF DESCRIPTION OF THE DRAWINGS

FIG. 1 is a front isometric view of the preferred embodiment of the system of the present invention.

FIG. 2 is a rear isometric view of the preferred embodiment of the system of the present invention.

FIG. 3 is an isometric view of the preferred embodiment of the system worn on the head of a user.

FIG. 4 is a diagram illustrating the operation of one embodiment of the infrared imaging system of the present invention to detect subcutaneous blood vessels.

FIG. 5A is an image of a human forearm showing unpolarized visible spectrum light reflected from the forearm and captured by a camera.

FIG. 5B is a raw image of the human forearm of FIG. 5A showing cross-polarized infrared spectrum light reflected from the forearm and captured by the CMOS camera of the preferred system of the present invention.

FIG. 5C is an enhanced image resulting from the operation of the computer program product of the present invention on the raw image of the human forearm of FIG. 5B.

FIG. 6 is a flow diagram of the preferred method of using the system to aid in detecting occlusions and leakages in subcutaneous blood vessels in accordance with the invention.

### DETAILED DESCRIPTION OF THE INVENTION

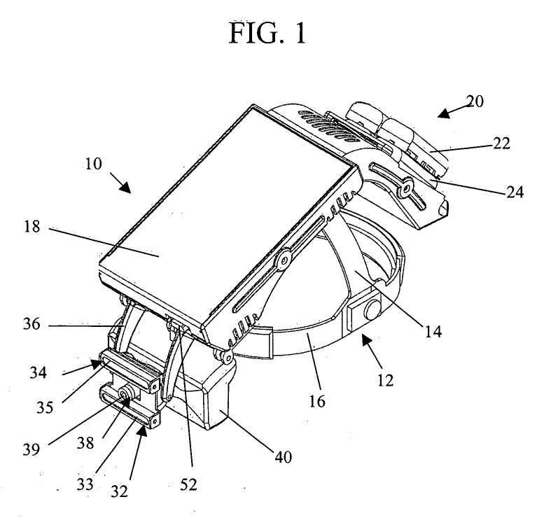

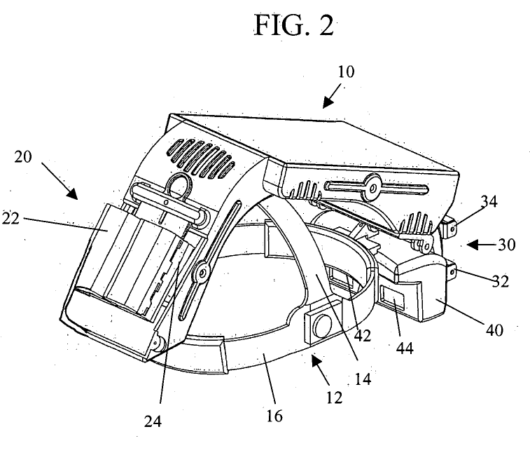

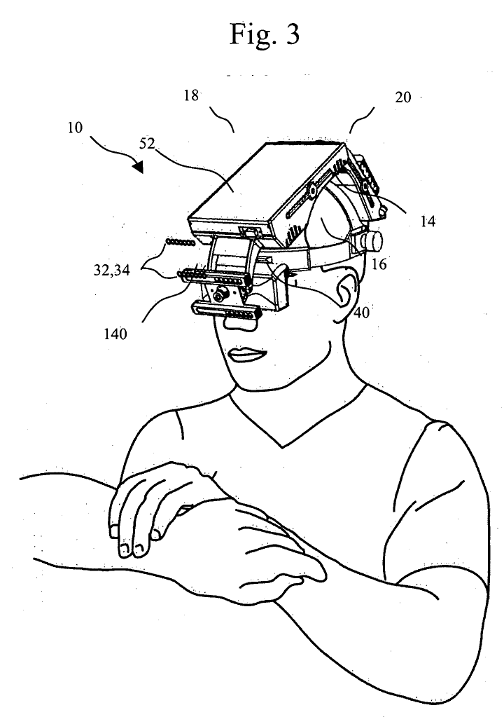

FIGS. 1-3 show the imaging system 10 used in the preferred method of the present invention. The preferred system 10 includes a headset 12 to which all system components are attached. The preferred headset 12 includes two plastic bands 14, 16 and a vertical band 14 connected to sides of a horizontal band 16. The vertical band 14, holding most of the system components, generally acts as a load-bearing member, while the horizontal band 16 is adjustable such that it snugly fits about the forehead of the person using the system.

A pivoting housing 18 is attached to the headband 12. The housing 18 is substantially hollow and is sized to house and protect a headset electronics unit 120 disposed therein. Attached to the housing 18 are a power supply 20, an image capture assembly 30, and an enhanced image display unit 40.

The power supply 20 for the headset electronics unit 120 preferably includes two rechargeable lithium ion batteries 22, which are connected to the electronics unit via a pair of battery terminals 24 attached to the rear of the housing 18. The rechargeable lithium ion batteries 22 are preferably of the same type commonly used with video camcorders, as these are readily available, are rechargeable without fear of memory problems, make the unit completely portable, and will provide sufficient power to the headset electronics unit 120 when two such batteries 22 are used. However, it is recognized that any power supply 20 known in the art to supply power to electronics, such as alternating current power plugs, may be employed to achieve similar results.

The image capture assembly 30 is powered thorough the headset electronics unit 120 and includes a pair of infrared emitters 32, 34, and a camera 38, or other infrared detector, disposed between the infrared emitters 32, 34. The infrared emitters 32, 34 and camera 38 are preferably attached to a common mounting surface 31 and are pivotally connected to a pair of extension arms 36 that extend from the housing 18. Mounting in this manner is preferred as it allows the emitters 32, 34 and camera 38 to be aimed at the proper target, regardless of the height or posture of the person wearing the headset. However, it is recognized that both could be fixedly attached to the headset, provided the relationship between the emitters 32, 34 and camera 38 remained constant.

The infrared emitters 32, 34 of the preferred embodiment are surface mount LEDs (light emitting diodes) that feature a built-in micro reflector. Light emitting diodes are particularly convenient when positioned about the head because they are found to generate less heat then conventional bulbs and do not require frequent changing. Further, surface mount LED's that emit infrared light through light shaping diffusers to provide uniform light and are readily adapted for attachment to a variety of other flat filter media. The preferred infrared emitters 32, 34 each utilize a row, or array, of such LED's in front of which is disposed a light shaping diffuser (not shown). Such emitters 32, 34 may be purchased from Phoenix Electric Co., Ltd., Torrance, Calif. First polarizing filters 33, 35 are mounted in front to the light shaping diffusers of each of the infrared emitters 32, 34. These polarizing filters 33, 35 are preferably flexible linear near-infrared polarizing filters, type HR, available from the 3M Corporation of St. Paul, Minn. In operation, the LED's are powered through the headset electronics unit 120 and emit infrared light, which passes through the light shaping diffuser 205 and the first polarizing filters 33, 35 to produce the polarized infrared light 215 that is directed upon the object to be viewed.

The camera 38 is adapted to capture the infrared light 230 reflected off of the object to be viewed and to provide this “raw image data” to the headset electronics unit 120. The preferred camera 38 is a monochrome CMOS camera that includes a high pass filter (not shown) that filters out all light outside of the infrared spectrum, including visible light. A CMOS camera is preferred as it produces pure digital video, rather than the analog video produced by the CCD cameras disclosed in the prior art, and is, therefore, not susceptible to losses, errors or time delays inherent in analog to digital conversion of the image. The CMOS camera is may be any number of such cameras available on the market, including the OMNIVISION® model OV7120, 640×480 pixel CMOS camera, and the MOTOROLA® model XCM20014. In the test units, the OMNIVISION® camera was used with good success. However, it is believed that the MOTOROLA® camera will be preferred in production due to its enhanced sensitivity to infrared light and the increased sharpness of the raw image produced thereby.

A camera lens 240 is preferably disposed in front of the camera 38. This camera lens 240 is preferably an optical lens that provides an image focal length that is appropriate for detection by the camera 38, preferably between six inches and fourteen inches, eliminates all non-near IR light, and reduces interference from other light signals. The preferred camera lens 240 is not adjustable by the user. However, other embodiments of the invention include a camera lens 240 that may be adjusted by the user in order to magnify and/or sharpen the image received by the camera 38. Still others eschew the use of a separate camera lens 240 completely and rely upon the detection of unfocused light by the camera 38, or other infrared detector.

A second linear polarizing filter 39 is disposed in front of the lens 240 of the camera 38. This second polarizing filter 39 is preferably positioned so as to be perpendicular to the direction of polarization through the first polarizing filters 33, 35 in front of the infrared emitters 32, 34, effectively cross polarizing the light detected by the camera 38 to reduce spectral reflection. The polarizing filter 39 was selected for its high transmission of near-infrared light and high extinction of cross-polarized glare. Such polarizer may be purchased from Meadowlark Optics, Inc. of Frederick, Colo. under the trademark VERSALIGHT®.

The camera 38 is in communication with the headset electronics unit 120 and sends the raw image data to the unit for processing. The headset electronics unit includes the electronics required to supply power from the power supply 20 to the image capture assembly 30, and an enhanced image display unit 40, and the compatible digital processing unit 122 which accepts the raw image data from the camera 38, enhances the raw image, and sends an output of the enhanced image to the enhanced image display unit 40 and, optionally, to an interface 52. In the preferred embodiment, this interface 52 is standard VGA output 52. However, interface 52 may be any electronic data I/Q interface capable of transmitting and receiving digital data to and from one or more input or output devices, such as an external monitor, external storage device, peripheral computer, or network communication path.

The preferred digital signal-processing unit 122 is a digital media evaluation kit produced by ATEME, Ltd SA, Paris, France under model number DMEK6414, which uses a Texas Instruments TMS320C6414 digital signal processor. This processing unit 122 is preferably programmed with an embodiment of the computer program means described in the applicants' co-pending U.S. patent application Ser. No. 10/760,051, in order to enhance the images. The image enhancement algorithms embodied in the computer program means utilize several elemental processing blocks, including (1) Gaussian Blurring a raw image with a kernel radius of 15, (2) adding the inverse Gaussian-blurred image to the raw image, and (3) level adjusting the result to use the entire dynamic range. Image enhancement is performed in a series of steps, which are coded into a computer program that runs on digital signal processor 120. The programming languages are typically C language and assembly language native to digital signal processor 120. An example algorithm is as follows:

```
    ON device startup
    BEGIN
      Perform Initialization of Blur Kernel
    END
      WHILE device = ON
    BEGIN
      Acquire digital image data from the camera into RAM buffer
      Save non-enhanced copy of the image data into another
      RAM buffer
      Perform 2D transform of image data in first RAM buffer into the
frequency domain
      Perform smoothing of transformed image data USING Blur Kernel
      Perform 2D inverse transform of smoothed image data into the
      spatial domain
      Perform inversion of the smoothed image data
      Perform add the inverted image data to the non-enhanced copy of
      the image data
      Perform contrast stretching
      Perform gamma enhancement.
      Send the enhanced image data to the display buffer
    END
```

However, it is understood that other systems may use different means for similarly enhancing such images in near real-time and, therefore, it is understood that all embodiments of the invention need not include this program product or perform the methods described in the above referenced patent application.

The enhanced image is outputted from the processing unit to the enhanced image display unit 40. The preferred display unit 40 is distributed by i-O Display Systems of Sacramento, Calif., under the trademark I-Glasses VGA. This display unit 40 includes a binocular display that includes a pair of LCD screens in front of which are disposed a pair of optical lenses 42, 44 that allow the focal length to be adjusted for ease of viewing. The preferred optical lenses 42, 44 provide image depth perception compensation to the user when the system 10 is used in a bifocal mode. That is, when the user views the body target area via display 150, the optical lenses 42, 44 ensure that the image appears similarly sized and distanced as when the user views the target area without using display 40. However, it is understood that a monocular display unit 40 having no such focal length adjustment could likewise be used. The preferred display unit 40 also includes an on-screen display that is not currently used, but may be used in the future to show what enhancement option has been chosen by the user.

The system 10 may be used in a total immersion mode, in which the user focuses on the target area by using exclusively display 40. Alternatively, the system 10 may be used in a bifocal mode, in which the user views the body target area via a combination of display 40 and the naked eye. In bifocal mode, the user alternates between viewing the enhanced and non-enhanced image views of the body target area, by directing his/her gaze upward to display 40 or downward toward the body target area and away from display 150.

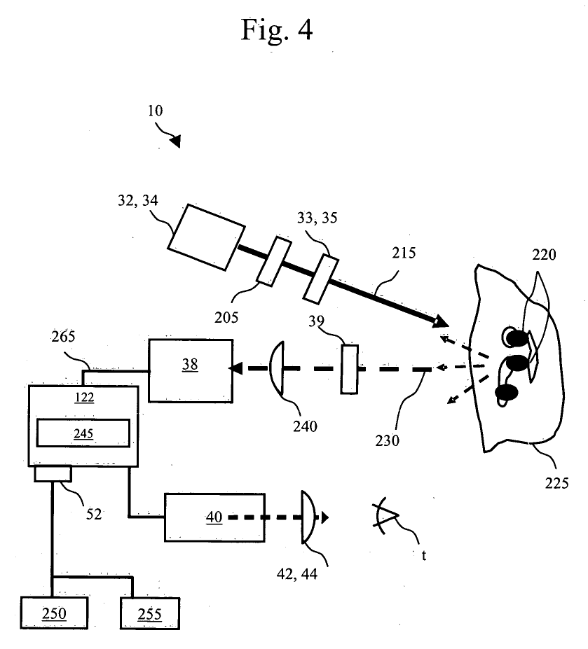

FIG. 4 illustrates one embodiment of the infrared imaging system 10 used to view subcutaneous blood vessels 220, such as arteries, veins, and capillary beds, which are present under the surface 225 of normal human skin. The infrared imaging system 10 described in connection with FIG. 4 includes all of the features of the preferred embodiment described above, in addition to including a camera lens 240, image data storage means 445, a data input 250, and data output 255.

Image data storage means 245 is any means of digital data storage that is compatible with digital signal processor 120 and may be used to store multiple enhanced and/or unenhanced images for future viewing. Examples of such image data storage are random access memory (RAM), read-only memory (ROM), personal computer memory card international association (PCMCIA) memory card, and memory stick. Depending on memory size, hundreds or thousands of separate images may be stored on the image data storage means 245.

Data output 250 is any external device upon which the image data produced by digital signal processor 120 may be viewed, stored, or further analyzed or conditioned. Examples of data output 250 devices include external video displays, external microprocessors, hard drives, and communication networks. Data output 250 interfaces with digital signal processor 120 via interface 52.

Data input 255 is any device through which the user of the system 10 inputs data to digital signal processor 122 in selecting, for example, the appropriate enhancement algorithm, adjusting display parameters, and/or choosing lighting intensity levels. Examples of data input 255 devices include external keyboards, keypads, personal digital assistants (PDA), or a voice recognition system made up of hardware and software that allow data to be inputted without the use of the user's hands. Data input 255 may be an external device that interfaces with digital signal processor 120 via interface 52, or may be integrated directly into the computing unit.

Digital data path 265 is an electronic pathway through which an electronic signal is transmitted from the camera 38 to the digital signal processor 122.

In operation, the infrared imaging system 10 is powered on and the infrared emitters 32, 34 produce the necessary intensity of IR light, at 850 nm and 950 nm wavelengths, required to interact and reflect from oxyhemoglobin and deoxyhemoglobin contained within normal blood and, alternatively, the necessary intensity and wavelengths of IR light to interact with the infrared viewable substance to be injected within the blood vessel. The resulting light path passes through diffuser system 205, where it is dispersed into a beam of uniform incident light 215 of optimal intensity and wavelength. Incident light 215 passes through first polarizers 33, 35, which provide a first plane of polarization. Polarization of incident light 215 reduces the glare produced by visible light by reflection from skin surface 225. Incident light 215 is partially absorbed by the oxyhemoglobin and deoxyhemoglobin, and/or the infrared visible substance, that is contained with subcutaneous blood vessels 220 and, thus, produces reflected light 230.

Reflected light 230 passes through second polarizer 39, which provides a second plane of polarization. The second plane of polarization may be parallel, orthogonal, or incrementally adjusted to any rotational position, relative to the first plane of polarization provided by first polarizers 33, 35. Reflected light 230, passes through first lens 240, which provides an image focal length that is appropriate for detection by the camera 38, eliminates all non-near IR light, and reduces interference from other light signals.

Camera 38 detects reflected light 230 and converts it to an electronic digital signal by using CCD, CMOS, or other image detection technology. The resulting digital signal is transmitted to digital signal processor 122 via digital signal path 265. Digital signal processor 122 utilizes a number of algorithms to enhance the appearance of objects that have the spatial qualities of blood vessels, so that the user can distinguish blood vessels easily from other features when viewed on display 40. Such enhancement might include, for example, image amplification, filtering of visible light, and image analysis. The resulting digital signal is transmitted to display 40 via digital signal path 265, where it is rendered visible by LCD, CRT, or other display technology. Additionally, the resulting digital signal may be outputted to an external viewing, analysis, or storage device via interface 52. The image produced by display 40 is then corrected for depth perception by second lens 260, such that, when the user views the body target area via display 40, the image appears similarly sized and distanced as when the user views the target area with the naked eye.

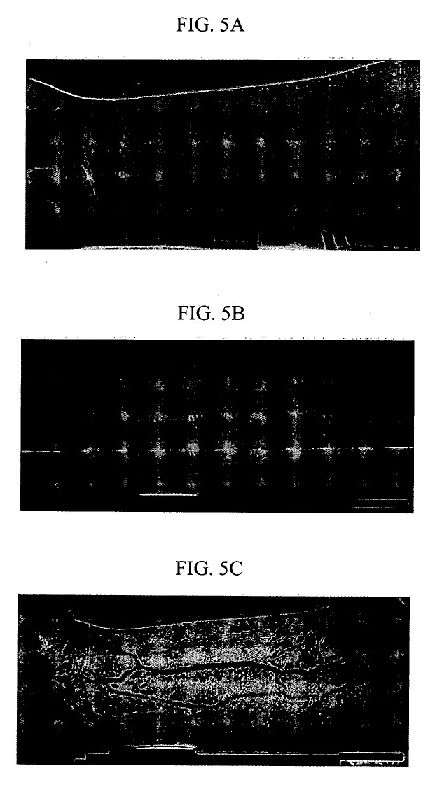

FIGS. 5A, 5B and 5C demonstrate the image enhancement produced by the system of the present invention. FIG. 5A is a photograph of a human forearm using light from the visible spectrum. As seen from this photograph, it is difficult to locate the veins upon visual inspection. FIG. 5B is a raw image of the same human forearm sent from the image capture assembly 30 of the present invention to the processing unit. The veins in this image are considerably more visible than those in FIG. 5A. However, they are not sufficiently dark and well defined to allow easy location of the veins during venepuncture. FIG. 5C is an enhanced image using the image enhancement process of the present invention. As can be seen from this figure, the veins are very dark and, therefore, are easily located for venepuncture.

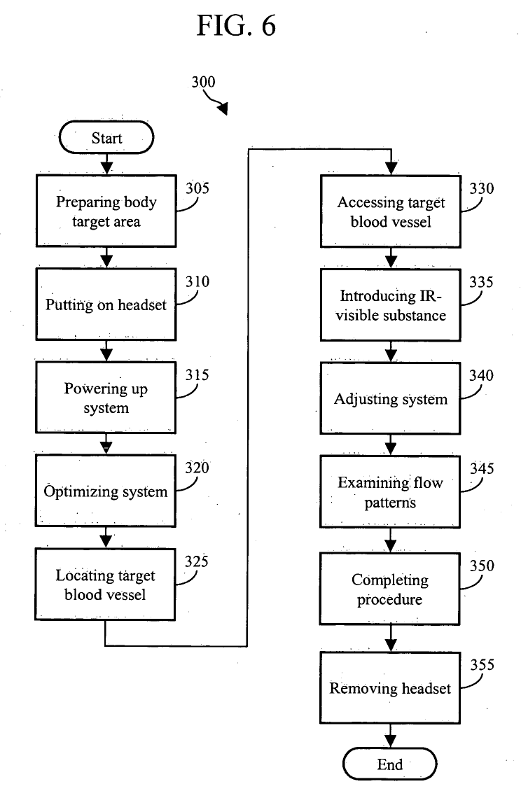

FIG. 6 illustrates a flow diagram of a preferred method 300 of using the system 10 to aid in the detection and diagnosis of blood vessel occlusions and leakages in accordance with the invention. The preferred method 300 includes the steps of:

Step 305: Preparing Body Target Area

In this step, a user, such as a medical practitioner (e.g. doctor, nurse, or technician), prepares the patient's body target area for injection by using standard medical practices. This might include, for example, positioning the target body area (e.g., arm), applying a tourniquet, swabbing the target area with disinfectant, and palpating the target area. Method 300 then proceeds to step 310.

Step 310: Putting on the Headset 12

In this step, the user places the headset 12 on his/her head and adjusts head mount 16 for size, comfort, and a secure fit. Method 300 then proceeds to step 315.

Step 315: Powering up the System

In this step, the user powers up the system 10, by activating a switch controlling the power source 20. Method 300 proceeds to step 320.

Step 320: Optimizing the System

In this step, the user uses data input 255 to adjust various parameters of the system 10, including specifying the appropriate digital signal processor 122 algorithms (according to, for example, the patient's body type, pigmentation, age), intensity levels of the infrared emitters 32, 34, wavelengths of light to be produced, and/or parameters for the images to be viewed on the display 40. Method 300 then proceeds to step 325.

It should be noted that Steps 310, 315, and 320 may be performed in any order, e.g., the user may power up the system 10 and optimize it, prior to putting it on. Further, it is recognized that optimizing step 320 may be eliminated altogether, with settings being preset at the factory.

Step 325: Locating Target Blood Vessel

In this step, the user searches non-invasively for the desired target blood vessel(s) (e.g., vein, artery, or capillary bed), by directing the incident light 215 from the infrared emitters 32, 34 on the body target area, viewing the target area on display 40, and focusing the camera lens 240 on the skin surface 225. As viewed on display 40, the target blood vessel(s) will be visually enhanced, i.e., appear darker than the surrounding tissue, which enables the user to insert a hypodermic needle more accurately and rapidly, in order to gain IV access for injection or blood withdrawal. Because of the hands-free operation of the system 10, the user is free to handle the body target area with both hands, for stability, further palpation, and cleansing, for example. Using the system 10 in a bifocal mode, the user may look down from display 40 to see the body target area as it appears under normal, non-enhanced conditions. Second lens 260 corrects the image displayed on display 40 for depth perception differences between the enhanced and non-enhanced images. Method 300 proceeds to step 330.

Step 330: Accessing Target Blood Vessel

In this step, the user, by utilizing either his/her naked eye or the enhanced image appearing on display 40, pierces skin surface 225 with catheter or needle tip and introduces the catheter or needle into the target blood vessel. By using the enhanced image of the target blood vessel displayed via display 40, the user is able to access the appropriate blood vessel more accurately and rapidly and, thus, save time and money and reduce the patient's physical and emotional pain and trauma. Method 300 proceeds to step 335.

Step 335: Introducing an IR-Visible Substance

In this step, the user introduces an IR-visible substance, such as indocyanine green into the target blood vessel by injecting it with a hypodermic needle or establishing an IV drip, for example. The amount of IR-visible substance introduced depends on the diagnostic application and monitoring period of method 300 and, therefore, is determined by the medical practitioner. It is noted that, in embodiments of method 300 in which no catheter or needle is used but, instead, a high velocity jet of IR-visible substance is used to introduce the substance into the blood vessel, that the accessing and introducing steps are combined into a single step. Method 300 proceeds to step 340.

Step 340: Adjusting the System

In this optional step, the user uses data input 255 to optimize the system 10 in order to better view the IR-opaque substance introduced in step 335. This may include an adjustment of digital signal processor 122 algorithms, intensity levels and/or wavelengths light emitted by the infrared emitters 32, 34, and parameter of the display 40, such as contrast and focal length, or other parameters of the system 10. In some embodiments, either the optimizing step 320 and the adjusting step 340 includes the step of optimizing or adjusting the system to rapidly cycle the IR light provided in order to allow both blood and the IR visible substance to be viewed. Method 300 proceeds to step 345.

Step 345: Examining Flow Patterns

In this step, the user, utilizing the enhanced image appearing on display 40, examines the flow patterns of the IR-opaque substance introduced in step 335. As viewed on display 40, the IR-opaque substance will be visually enhanced, i.e., appear darker than the surrounding tissues and structures. For example, the user may identify a vessel obstruction (i.e., blood clot) by observing an absence of IR-opaque substance through the target blood vessel. In another example, the user may observe a leakage (i.e., internal bleeding) by observing the IR-opaque substance flowing outside of the target blood vessel. In yet another example, by observing a restricted flow through a particular area of the target blood vessel, the user may observe a blood vessel narrowing. Each type of condition is identifiable by a resulting unique flow pattern.

Flow pattern sequences may be recorded on data storage 245 and reviewed on display 40 (or external device) at a later time. Upon playback, digital signal processor 122 may be adjusted to alter flow pattern sequences by speeding the sequences up, slowing the sequences down, or otherwise modifying flow pattern sequences, in order to aid the user in viewing and diagnosing. Method 300 proceeds to step 350.

Step 350: Completing Procedure

In this step, the user completes the injection, examination, and/or diagnosis procedure, by using standard medical practices. This may include withdrawing the cannula and cleansing the injection area, for example. Method 300 proceeds to step 355.

Step 355: Removing the Headset 12

In this step, the user removes the headset 12 from his/her head and powers off the system 10. Alternatively, the user prepares additional patients/body target areas for imaging and injection. Method 300 ends.

As noted above, that the present invention is not limited to diagnosis and monitoring of occlusions using the preferred system 12, but rather may be performed using any IR imaging system that includes at least one infrared emitter an infrared detector, a computing unit, a display device, and a power source. Due to the injection of a highly visible substance within the blood vessel, and the fact that the step 345 of examining flow patterns does not require that real time images be provided to the display, the imaging system used to perform the method may not enhance images, or provide images to the display in substantially real time. Therefore, in these embodiments, steps 305, 310, 320, and 355 may be omitted, and step 325 may be performed after the blood vessel has been accessed and the IR-visible substance has been injected.

Method 300 may be used on a one-time basis to diagnose suspected IV disorders or may be used multiple times to monitor the progression of IV disorders over extended time periods. In cases for which ongoing monitoring is determined appropriate by the medical practitioner, he/she determines an examination schedule and duration, and method 300 is repeated at regular intervals (e.g., every 30 days), until the time period has elapsed or until no further monitoring is needed.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. A method for detecting occlusions and leakages in subcutaneous blood vessels with the aid of an infrared imaging system, wherein the imaging system comprises, at least one infrared emitter, an infrared detector, a computing unit in communication with the infrared detector, a display in communication with the computing unit, and a power source;
wherein said method comprises the steps of:
preparing a body target area;
supplying power from the power source to the infrared emitter, infrared detector, computing unit, and display of the imaging system, such that infrared light is emitted by the infrared emitter, reflected infrared light is received by the infrared detector and converted into signals sent to the computing unit, the computing unit accepts the signals and outputs image data to the display, and the display displays the images;
accessing a target blood vessel;
introducing an IR-opaque substance into the target blood vessel;
locating the target blood vessel such that images of the target blood vessel are captured by the infrared detector and displayed on the display; and
examining flow patterns of the IR-visible substance through the target blood vessel by viewing the images of the target blood vessel on the display of the imaging system.

2. The method of claim 1 wherein said step of examining flow patterns comprises examining images displayed on the display to determine the presence of an occlusion of the blood vessel by observing an absence of IR-opaque substance through the target blood vessel.

3. The method of claim 1 wherein said step of examining flow patterns comprises examining images displayed on the display to determine the presence of a leakage through the target blood vessel by observing the IR-opaque substance flowing outside of the target blood vessel.

4. The method of claim 1 wherein said step of examining flow patterns comprises examining images displayed on the display to determine the presence of a narrowing of a target blood vessel by observing a restricted flow of the IR-opaque substance through a particular area of the target blood vessel.

5. The method of claim 1 wherein the computing unit of the infrared imaging system enhances images of the target blood vessel before outputting the images to the display;
wherein the locating step is performed before the accessing step; and
wherein said accessing step comprises the step of viewing an enhanced image of the target blood vessel on the display of the imaging system and piercing the target blood vessel with the aid of the enhanced image.

6. The method of claim 5 wherein said locating step comprises the steps of:
directing incident light from the infrared emitters on a target area of a surface of a skin; and
viewing the enhanced image of blood vessels located beneath the target area on the display.

7. The method of claim 6 wherein the display of the imaging system comprises an optical lens disposed between the display and an eye of a user and wherein said locating step further comprises the steps of:
viewing the unenhanced image on the target area of the skin; and
adjusting the optical lens to correct the enhanced image displayed on the display for depth perception differences between the enhanced image and the unenhanced image.

8. The method of claim 6 wherein said step of locating a target blood vessel further comprises the steps of:
viewing the unenhanced image on the target area of the skin;
adjusting the display to correct the enhanced image displayed on display for depth perception differences between the enhanced image and the unenhanced image.

9. The method of claim 5 further comprising the step of optimizing the system, wherein the computing unit comprises a digital signal processor and a memory, wherein the system comprises a data input, and wherein said step of optimizing the system comprises the step of using the data input to specify an enhancement algorithm stored in memory to be used by the digital signal processor to generate the enhanced image.

10. The method of claim 9 wherein said step of optimizing the system further comprises the step of selecting an enhancement algorithm based upon a factor selected from a group consisting of a body type, pigmentation, age of the patient, and characteristics of the IR-visible substance introduced into the target blood vessel.

11. The method of claim 9 wherein said step introducing an IR-visible substance into the target blood vessel comprises introducing an IR-opaque substance into the target blood vessel, and wherein said step of optimizing the system further comprises the step of selecting an enhancement algorithm based upon and characteristics of the IR-opaque substance.

12. The method of claim 9 wherein said step of optimizing the system further comprises the step of using said data input to adjust at least one of an intensity level of the at least one infrared emitter and a wavelength of infrared light emitted by said at least one infrared emitter.

13. The method of claim 1 wherein the imaging system further comprises a headset to which the infrared emitter, infrared detector, computing unit, and display are attached, and wherein said method further comprises the step of disposing the headset on a head of a user.

14. The method of claim 9 wherein said step of examining flow patterns comprises examining images displayed on the display to determine the presence of an occlusion of the blood vessel by observing an absence of IR-opaque substance through the target blood vessel.

15. The method of claim 9 wherein said step of examining flow patterns comprises examining images displayed on the display to determine the presence of a leakage through the target blood vessel by observing the IR-opaque substance flowing outside of the target blood vessel.

16. The method of claim 1 wherein said step of examining flow patterns comprises examining images displayed on the display to determine the presence of a narrowing of a target blood vessel by observing a restricted flow of the IR-opaque substance through a particular area of the target blood vessel.

17. The method of claim 9 wherein the computing unit of the infrared imaging system enhances images of the target blood vessel before outputting the images to the display;
wherein the locating step is performed before the accessing step; and
wherein said accessing step comprises the step of viewing an enhanced image of the target blood vessel on the display of the imaging system and piercing the target blood vessel with the aid of the enhanced image.

18. The method of claim 17 wherein said locating step comprises the steps of:
directing incident light from the infrared emitters on a target area of a surface of a skin; and
viewing the enhanced image of blood vessels located beneath the target area on the display.

19. The method of claim 18 wherein the display of the imaging system comprises an optical lens disposed between the display and an eye of a user and wherein said locating step further comprises the steps of:
viewing the unenhanced image on the target area of the skin; and
adjusting the optical lens to correct the enhanced image displayed on the display for depth perception differences between the enhanced image and the unenhanced image.

20. The method of claim 18 wherein said step of locating a target blood vessel further comprises the steps of:
viewing the unenhanced image on the target area of the skin;
adjusting the display to correct the enhanced image displayed on display for depth perception differences between the enhanced image and the unenhanced image.

21. The method of claim 17 further comprising the step of optimizing the system, wherein the computing unit comprises a digital signal processor and a memory, wherein the system comprises a data input, and wherein said step of optimizing the system comprises the step of using the data input to specify an enhancement algorithm stored in memory to be used by the digital signal processor to generate the enhanced image.

22. The method of claim 21 wherein said step of optimizing the system further comprises the step of selecting an enhancement algorithm based upon a factor selected from a group consisting of a body type, pigmentation, age of the patient, and characteristics of the IR-visible substance introduced into the target blood vessel.

23. The method of claim 21 wherein said step introducing an IR-visible substance into the target blood vessel comprises introducing an IR-opaque substance into the target blood vessel, and wherein said step of optimizing the system further comprises the step of selecting an enhancement algorithm based upon and characteristics of the IR-opaque substance.

24. The method of claim 21 wherein said step of optimizing the system further comprises the step of using said data input to adjust at least one of an intensity level of the at least one infrared emitter and a wavelength of infrared light emitted by said at least one infrared emitter.

25. The method of claim 17 further comprising the step of adjusting the system, wherein said adjusting step is performed after said introducing step.

26. The method of claim 25 wherein the computing unit comprises a digital signal processor and a memory, wherein the system comprises a data input, and wherein said step of adjusting the system comprises the step of using the data input to specify an enhancement algorithm stored in memory to be used by the digital signal processor to generate the enhanced image.

27. The method of claim 25 wherein the computing unit comprises a digital signal processor and a memory, wherein the system comprises a data input, and wherein said step of adjusting the system comprises the step of using the data input to alter the mode operation of the infrared emitters.

28. The method of claim 27 wherein said step of using the data input to alter the mode of operation of the infrared emitters comprises using the data input to alter the intensity level of the infrared emitters.

29. The method of claim 27 wherein said step of using the data input to alter the mode of operation of the infrared emitters comprises using the data input to alter the wavelengths of light emitted by the infrared emitters.

30. The method of claim 25 wherein the computing unit comprises a digital signal processor and a memory, wherein the system comprises a data input, and wherein said step of adjusting the system comprises the step of using the data input to alter parameters of the display.

31. The method of claim 1 wherein the imaging system further comprises data storage means for storing multiple images and wherein said method further comprises the step of recording a sequence of images showing a flow pattern on the data storage means.

32. The method of claim 31 wherein the computing unit of the imaging system comprises a digital signal processor programmed with an algorithm to adjust a playback of the sequence of enhanced images stored in the data storage means and wherein said method further comprises the step of adjusting the playback of the sequence of enhanced images stored in the data storage means.

33. The method of claim 17 wherein the imaging system further comprises data storage-means for storing multiple enhanced images and wherein said method further comprises the step of recording a sequence of enhanced images showing a flow pattern on the data storage means.

34. The method of claim 33 wherein the computing unit of the imaging system comprises a digital signal processor programmed with an algorithm to adjust the playback of the sequence of enhanced images stored in the data storage means.

35. The method of claim 17 further comprising the steps of removing the headset and powering off the system.
